(19) United States
(12) Patent Application Publication
(10) Pub. No.: US 2019/0298529 A1
(43) Pub. Date: Oct. 3, 2019

(54) ORTHOPAEDIC HIP PROSTHESIS SURFACE PROTECTION SYSTEM AND METHOD

(71) Applicant: DePuy Synthes Products, Inc., Raynham, MA (US)

(72) Inventors: Justin D. Grostefon, Warsaw, IN (US); Larry G. McCleary, Warsaw, IN (US); Jeffrey A. McAnelly, Warsaw, IN (US)

(21) Appl. No.: 15/941,891

(22) Filed: Mar. 30, 2018

(51) Int. Cl. A61F 2/36

(52) U.S. Cl. CPC A61F 2/3609; A61F 2/367; A61F 2002/365; A61F 2002/3652; A61F 2002/3625

(57) ABSTRACT

An orthopaedic system includes a femoral stem assembly having a protective casing or shell that protects one or more mating surfaces of the femoral stem component from incidental damage. The system includes a femoral head assembly having a protective casing or shell that protects one or more mating surfaces of the femoral head component from incidental damage. The system may also include a surgical instrument that is configured to detach or remove the protective casings from the prosthetic components of the femoral stem assembly and the femoral head assembly and secure the femoral stem component to the femoral head component. Methods of use and performing an orthopaedic surgical procedure are also disclosed.

### TECHNICAL FIELD

The present disclosure relates generally to orthopaedic hip prostheses and, more particularly, to surgical systems used to protect engagement surfaces of an orthopaedic hip prosthesis.

### BACKGROUND

Joint arthroplasty is a well-known surgical procedure by which a diseased or damaged natural joint is replaced by a prosthetic joint. The prosthetic joint may include a prosthesis that is implanted into one or more of the patient's bones. Many hip prostheses include a femoral prosthesis that is implanted into a patient's femur. A femoral prosthesis typically includes a femoral stem component that is received in the medullary canal of the patient's femur and a semi-spherical femoral head component that bears against the patient's acetabulum or a prosthetic replacement acetabular cup.

The elongated stem component may be cemented into the medullary canal or may have a surface conducive for allowing the bone to heal directly to the implant. In some prostheses, the head component is attached to a neck of the stem component via a tapered trunnion. An exemplary tapered trunnion is shown and described in U.S. Pat. No. 9,615,927, which is incorporated herein by reference. The acetabulum of the patient may also be reamed to receive a shell and a liner. A polyethylene, metal or ceramic liner with a metal shell is inserted into the acetabulum and acts as a socket for receiving the head component.

The implants of the hip prosthesis are often included in orthopaedic systems that include surgical instruments designed to facilitate implantation of the implants into the patient's bones. Such surgical instruments may include cutting blocks, bone saws, assembly tools, and so forth configured to prepare the patient's bones to receive the implants and/or assist the surgeon during selection and placement of the implants in the patient's bones.

### SUMMARY

According to one aspect of the disclosure, an orthopaedic system is disclosed. The system includes a femoral stem assembly having a protective casing or shell that protects one or more mating surfaces of the femoral stem component from incidental damage. In some embodiments, the mating surface is a tapered trunnion surface of the femoral stem component. The system includes a femoral head assembly having a protective casing or shell that protects one or more mating surfaces of the femoral head component from incidental damage. In some embodiments, the mating surfaces of the femoral head include the tapered inner surface of the femoral head component and/or the semi-spherical outer surface of the femoral head component. The orthopaedic system may include a surgical instrument configured to be coupled to the femoral stem assembly and the femoral head assembly. The surgical instrument may be operated to detach the protective casings from the prosthetic components of the femoral stem assembly and the femoral head assembly and secure the femoral stem component to the femoral head component.

According to another aspect, an orthopaedic system comprises a femoral head component including a semi-spherical articular surface, a distal surface, and a tapered bore formed in the distal surface. The system also comprises a femoral stem component configured to be received in a proximal end of a patient's surgically-prepared femur. The stem component comprises a neck including a trunnion sized to be positioned in the tapered bore of the femoral head component and an elongated body extending distally from the neck. The system further comprises a first removable shell extending over the trunnion of the femoral stem component. The first removable shell includes a number of seams extending parallel to a longitudinal axis of the trunnion. Each seam is configured to facilitate separation of the first removable shell into a number of shell segments to permit the first removable shell to be removed from the trunnion. The tapered trunnion of the femoral stem component is configured to be taper locked to the femoral head component when the first removable shell is removed from the tapered trunnion.

In some embodiments, the orthopaedic system may further comprise a second removable shell coupled to the femoral head component extending over the semi-spherical articular surface and a distal opening of the tapered bore The first removable shell may include a sleeve extending over the trunnion of the femoral stem component and a proximal tab extending outwardly from the sleeve. The proximal tab may be configured to pierce the second removable shell.

Additionally, in some embodiments, the second removable shell may include an inner flange configured to engage the proximal tab of the first removable shell to facilitate separation of the first removable shell into the number of shell segments.

In some embodiments, the second removable shell may further include a distal component coupled to the distal surface of the femoral head component and a flexible sheath encasing the femoral head component and the distal component. The distal component may include a central passageway aligned with the tapered bore of the femoral head component, the inner flange, which extends into the central passageway. The central passageway may be sized to receive the first removable shell and the tapered trunnion of the femoral stem component.

Additionally, in some embodiments, the proximal tab of the first removable shell may include a plurality of proximal tabs extending from the sleeve that are configured to pierce a surface of the flexible sheath covering the central passageway. Each proximal tab may be connected to a section of the sleeve positioned between a pair of seams.

In some embodiments, the inner flange may be an annular flange configured to engage the plurality of proximal tabs and extending around the central passageway of the distal component.

In some embodiments, the distal component of the second removable shell may include a plurality of component segments. Each component segment may include a pair of sidewalls extending along a longitudinal axis of the central passageway. Each sidewall of each component segment may be engaged with the sidewall of an adjacent component segment to form the distal component.

In some embodiments, the orthopaedic system may further comprise a surgical instrument configured to be coupled to the second removable shell and the elongated body of the femoral stem component. The surgical instrument may be operable to advance the second removable shell distally into contact with the proximal tab of the first removable shell to pierce the second removable shell.

In some embodiments, the second removable shell may include a distal component coupled to the distal surface of the femoral head component, a flexible sheath encasing the femoral head component and the distal component, and a proximal component extending outwardly from the flexible sheath. The distal component may include a central passageway aligned with the tapered bore of the femoral head component, and the central passageway may be sized to receive the first removable shell and the tapered trunnion of the femoral stem component. The proximal component may include a concave distal surface shaped to match a curvature of the semi-spherical articular surface of the femoral head component and a connector configured to be coupled to the surgical instrument.

In some embodiments, the first removable shell may include a body having a plurality of longitudinal segments connected via the number of seams. Each longitudinal segment may correspond to one of the number of shell segments.

In some embodiments, each longitudinal segment may have a first thickness and each seam may have a second thickness that is less than the first thickness.

According to another aspect, an orthopaedic system comprises a femoral head component including a semi-spherical articular surface, and a removable shell coupled to the femoral head component. The femoral head component also includes a distal surface. and a tapered bore formed in the distal surface. The removable shell comprises a distal component coupled to the distal surface of the femoral head component, a flexible sheath encasing the femoral head component and the distal component, and a proximal component extending outwardly from the flexible sheath. The distal component includes a central passageway aligned with the tapered bore of the femoral head component, and the proximal component includes a concave distal surface shaped to match a curvature of the semi-spherical articular surface of the femoral head component and a connector configured to be coupled to a surgical instrument.

In some embodiments, the distal component of the removable shell may include a plurality of component segments. Each component segment may include a pair of sidewalls extending along a longitudinal axis of the central passageway. Each sidewall of each component segment may be engaged with the sidewall of an adjacent component segment.

In some embodiments, the orthopaedic system may further comprise a femoral stem component configured to be received in a proximal end of a patient's surgically-prepared femur. The stem component may comprise a neck including a trunnion sized to be positioned in the tapered bore of the femoral head component and an elongated body extending distally from the neck. The central passageway of the removable shell may be sized to receive the trunnion of the femoral stem component.

In some embodiments, the removable shell may be a femoral head protective casing, and the orthopaedic system may further comprise a trunnion protective casing that extends over the trunnion of the femoral stem component. The trunnion protective casing may include a proximal tab configured to pierce the femoral head protective casing to permit the trunnion and the trunnion protective casing to enter the central passageway of the trunnion protective casing.

In some embodiments, the trunnion protective casing may include a plurality of longitudinal segments extending over the trunnion of the femoral stem component. The longitudinal segments may be connected via a number of seams, and the proximal tab may be one of a plurality of proximal tabs. Each proximal tab may extend proximally from a corresponding longitudinal segment.

Additionally, in some embodiments, the distal component of the femoral head protective casing may include an inner flange extending into the central passageway. The inner flange may be configured to engage the plurality of proximal tabs to separate the trunnion protective casing into the plurality of longitudinal segments to detach the trunnion protective casing from the trunnion.

According to another aspect of the disclosure, a method comprises selecting a femoral stem component and selecting a femoral head component covered by a second removable shell. The femoral stem component comprises a neck including a tapered trunnion covered by a first removable shell and an elongated body extending distally from the neck. The method further comprises aligning a distal end of the second removable shell with the tapered trunnion of the femoral stem component, positioning the second removable shell over the first removable shell and the tapered trunnion to engage a plurality of proximal tabs of the first removable shell with an inner flange of the second removable shell, advancing the second removable shell and the femoral head component distally to cause the proximal tabs to move distally and split the first removable shell into a number of shell segments, and moving the femoral head component distally to position the tapered trunnion in a tapered bore of the femoral head component.

In some embodiments, positioning the second removable shell over the first removable shell and the tapered trunnion may comprise piercing the distal end of the second removable shell with the plurality of proximal tabs, and moving the first removable shell and the tapered trunnion into a central passageway defined in the second removable shell.

In some embodiments, advancing the second removable shell and the femoral head component distally to cause the proximal tabs to move distally and split the first removable shell into the number of shell segments may further comprise separating a distal component of the second removable shell into a plurality of component segments. Each component segment may be coupled to a corresponding shell segment of the first removable shell.

In some embodiments, the method may further comprise detaching the first removable shell and the second removable shell from the femoral head component after the tapered trunnion is positioned in the tapered bore of the femoral head component. The first removable shell may be secured to the second removable shell via the engagement between the inner flange and the proximal tabs.

### BRIEF DESCRIPTION OF THE DRAWINGS

The detailed description particularly refers to the following figures, in which:

FIG. 1 is an exploded perspective view of an orthopaedic system;

FIG. 2 is perspective view of a protective casing positioned on a trunnion of a femoral stem component of the orthopaedic system of FIG. 1;

FIG. 3 is a cross-sectional side elevation view taken along the line 3-3 in FIG. 2;

FIG. 4 is a perspective view of the protective casing of FIG. 2;

FIG. 5 is a partial cross-sectional perspective view of another protective casing positioned on a femoral head component of the orthopaedic system of FIG. 1;

FIG. 6 is a perspective view of a proximal component of the protective casing of FIG. 5;

FIG. 7 is a perspective view of a component segment of a distal component of the protective casing of FIG. 5;

FIG. 8 is a rear elevation view of a distal component of the protective casing of FIG. 5;

FIG. 9 is a perspective view of an orthopaedic surgical instrument for use in assembling the femoral head component and the femoral stem component of the orthopaedic system of FIG. 1;

FIG. 10 is a perspective view of the orthopaedic system of FIG. 1 with the femoral head component and the femoral stem component aligned for assembly;

FIG. 11 is a cross-sectional side elevation view taken along the line 11-11 in FIG. 10;

FIG. 12 is a perspective view of the orthopaedic system of FIG. 1 with the trunnion of the femoral stem component and its trunnion protective casing inserted into a passageway of the femoral head protective casing;

FIG. 13 is a cross-sectional side elevation view taken along the line 13-13 in FIG. 12; and

FIG. 14 is a cross-sectional side elevation view similar to FIG. 13 showing the trunnion of the femoral stem component assembled with the femoral head component.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

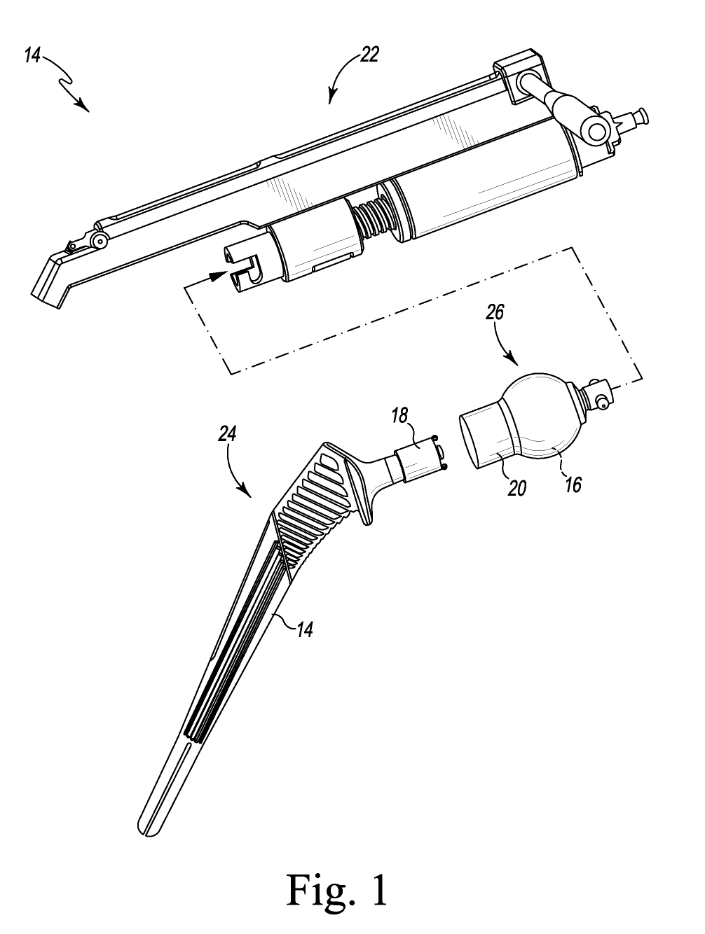

Referring now to FIG. 1, an orthopaedic system 10 includes a hip prosthesis configured for implantation into a patient's femur during a hip replacement surgical procedure. The hip prosthesis includes a femoral stem component 14 and a femoral head component 16 configured to be secured to the femoral stem component 14. Each of the prosthetic components 14, 16 are formed from implant-grade metallic materials such as, for example, cobalt chromium or stainless steel. In the illustrative embodiment, the prosthetic components 14, 16 are configured to be secured together via a taper lock, which is created between clean, dry, and undamaged mating surfaces of the respective prosthetic components 14, 16.

The orthopaedic system 10 also includes a number of protective casings 18, 20 that are configured to maintain the mating surfaces of the components 14, 16 in proper condition to form the taper lock. As described in greater detail below, the casings 18, 20 are removable shells that extend over portions of the prosthetic components 14, 16 and are configured to be detached during the process of securing the components 14, 16 together. The orthopaedic system 10 includes an assembly tool 22 configured to advance the femoral head component 16 onto the femoral stem component 14 to detach the casings 18, 20 and form the taper lock to secure the prosthetic components 14, 16 together during the hip replacement surgical procedure.

In their pre-surgical procedure configuration, the femoral stem component 14 and the protective casing 18 form a femoral stem assembly 24 that is assembled by, for example, the orthopaedic manufacturer. Similarly, the femoral head component 16 and the protective casing 20 form a femoral head assembly 26 that is separately assembled by, for example, the same orthopaedic manufacturer that makes the femoral stem assembly 24 or by a different manufacturer. It should be appreciated that the system 10 may include multiple assemblies 24, 26 of different sizes and configurations so that surgeons may select the assemblies 24, 26 that are the most appropriate for an individual patent's anatomy. In other embodiments, the system 10 may include only various assemblies 24 or only various assemblies 26 such that the corresponding femoral head or stem components do not include the protective casings.

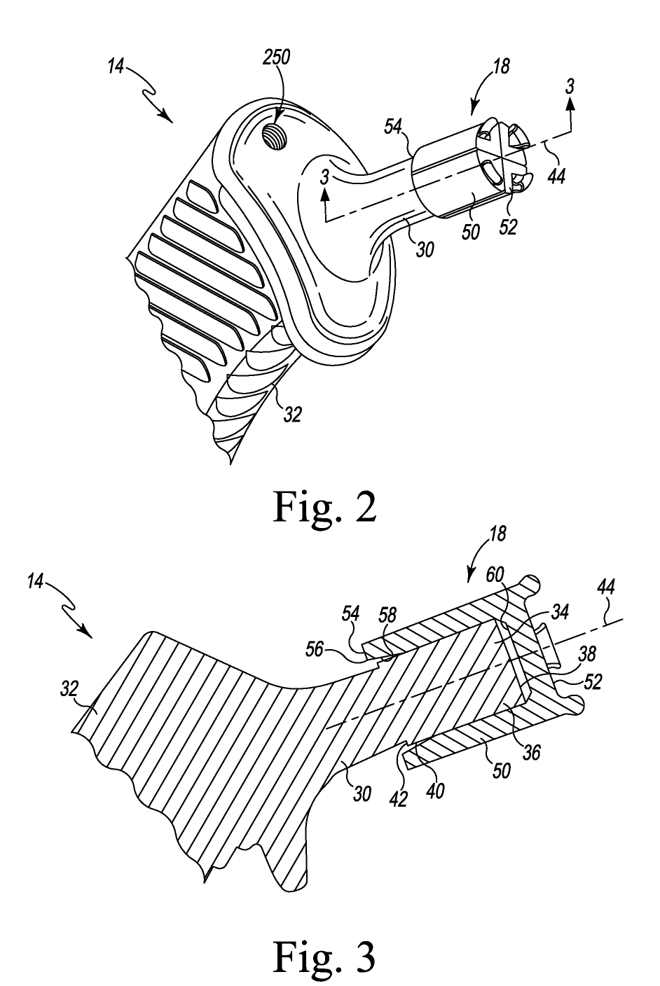

Referring now to FIGS. 2-3, the femoral stem component 14 includes a neck 30 and an elongated body 32 that extends distally from the neck 30 to a distal tip. The elongated body 32 is sized and shaped to be positioned in a surgically-prepared medullary canal of a patient's femur. The neck 30 is configured to extend medially and proximally away from the proximal end of the patient's femur when the elongated body 32 is properly positioned in the medullary canal. The neck 30 extends to a proximal tip 34, as shown in FIG. 3.

The femoral stem component 14 also includes a trunnion 36, which is formed at the proximal tip 34 and is configured to engage the femoral head component 16 to form the tapered lock. In the illustrative embodiment, the trunnion 36 includes an end surface 38 and a tapered outer surface 40 that extends distally from the end surface 38 to a distal edge 42 along a longitudinal axis 44 of the trunnion 36. In the illustrative embodiment, the protective casing 18 is positioned on the trunnion 36 to protect the trunnion from incidental damage and maintain it in a dry and clean condition prior to assembly with the femoral head component 16.

In the illustrative embodiment, the casing 18 includes a sleeve 50 that extends from a proximal end 52 over the surfaces 38, 40 of the trunnion 36 to a distal end 54 that is positioned distal of the distal edge 42 of the trunnion 36. The sleeve 50 includes an opening 56 in the distal end 54, and an inner wall 58 extends inwardly from the opening 56 to a base wall 60. The inner wall 58 is engaged with the tapered outer surface 40 of the trunnion 36 to secure the casing 18 to the femoral stem component 14.

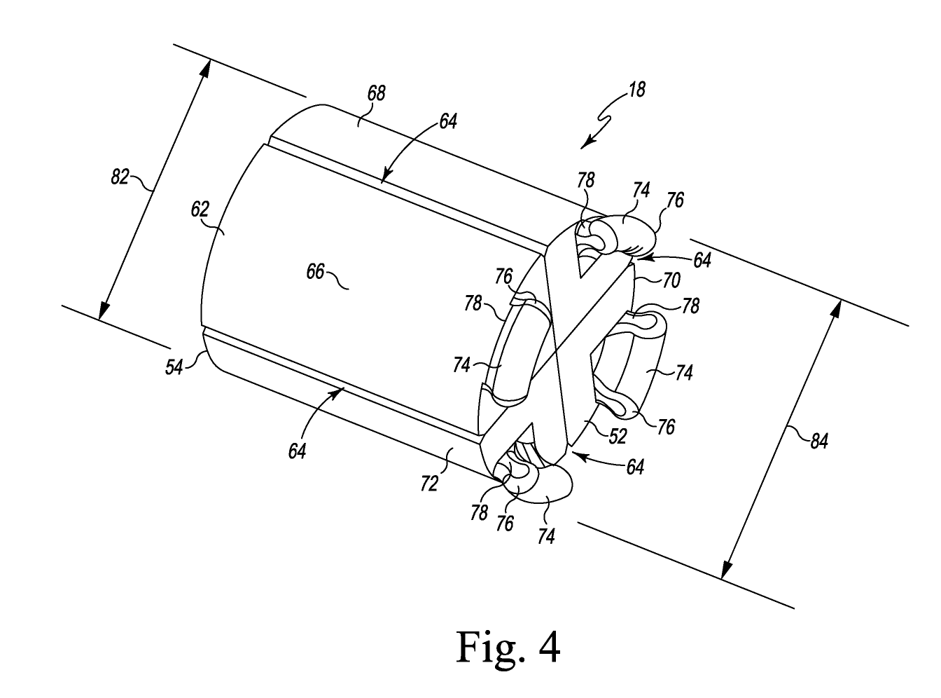

As shown in FIG. 4, the sleeve 50 has a curved outer surface 62 that extends between its ends 52, 54. The casing 18 also include a plurality of longitudinal seams 64 that extend from the proximal end 52 of the sleeve 50 to the distal end 54. In the illustrative embodiment, each seam 64 is a groove defined in the curved outer surface 62 of the sleeve 50, and each groove has a v-shaped cross-section. It should be appreciated that, as a result, the wall thickness of the sleeve 50 at the base (i.e., the bottom of each groove) of each seam 64 is less than the general wall thickness of the sleeve 50. The seams 64 also separate the sleeve 50 into a number of sleeve segments 66, 68, 70, 72, as described in greater detail below. It should be appreciated that in other embodiments the seams may take the form of, for example, a helical pattern. In some embodiments, the slots may only partially extend through the body 18. In other embodiments, the casing may use elastic or flexible materials which would not require seems yet would effectively function in a similar manner

The protective casing 18 also includes a number of proximal tabs 74 that extend outwardly from the proximal end 52 of the sleeve 50. As shown in FIG. 4, each tab 74 includes a curved head 76 and a stem 78 that connects the head 76 to the sleeve 50. In the illustrative embodiment, the tabs 74 are arranged around the outer edge of the sleeve 50 between the seams 64, with one tab 74 attached to each of the sleeve segments 66, 68, 70, 72. The tabs 74 cooperate to define an outer diameter 80 that is greater than the diameter 82 of the sleeve 50. It should be appreciated that in other embodiments the casing 18 may include additional tabs or fewer proximal tabs.

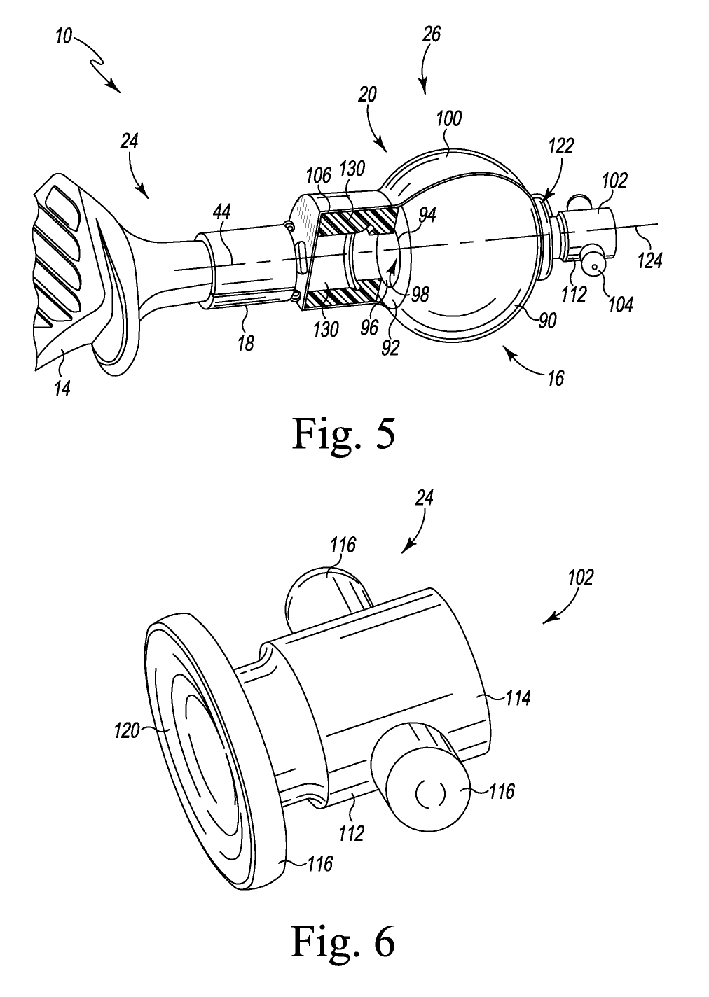

Referring now to FIG. 5, the orthopaedic system 10 also includes a femoral head component 16 that is encased within a femoral head protective casing 20 to form a femoral head assembly 26. The femoral head component 16 includes a planar distal surface 92 that is connected to a semi-spherical proximal surface 90, which is configured to articulate with an acetabular prosthesis (not shown) that is positioned in a patient's surgically-prepared acetabulum. An opening 94 is defined in the distal surface 92, and an inner wall 96 extends inwardly from the opening 94 define a bore 98 in the femoral head component 16. The inner wall 96 is curved and is tapered such that the bore 98 is a tapered bore. The taper of the inner wall 96 matches the taper of the outer surface 40 of the stem trunnion 36 such that the tapered bore 98 is sized to receive the trunnion 36. It should be appreciated that when the femoral head component 16 is advance over the trunnion 36 of the femoral stem component 14, engagement between the outer surface 40 of the trunnion 36 and the inner wall 96 of the head component 16 forms the taper lock that secures the prosthetic components 14, 16 together. In that way, the surface 40 and wall 96 act as mating surfaces for the prosthetic components 14, 16.

The femoral head protective casing 20 encases the femoral head component 16 to protect the semi-spherical proximal surface 90 and the inner wall 96 from incidental damage and maintain the femoral head component 16 in a dry and clean condition prior to assembly with the femoral stem component 14. In the illustrative embodiment, the protective casing 20 includes an outer sheath 100 that extends over the femoral head component 16 and a proximal component 102 that extends outwardly from the sheath 100. As described in greater detail below, the proximal component 102 includes a connector 104 configured to be coupled to the assembly tool 22 to attach the femoral head protective casing 20 (and hence the femoral head assembly 26) to the assembly tool. The protective casing 20 also includes a distal component 106 that engages the distal surface 92 of the femoral head component 16 and is encased with the femoral head component in the outer sheath 100.

It should be appreciated that the outer sheath 100 forms a packaging layer of the protective casing 20. The outer sheath 100 is illustratively formed from a flexible material such as, for example, polyurethane. The proximal component 102 is illustratively formed from polyethylene, but in other embodiments may be formed from any suitable plastic material. In still other embodiments, the component 102 may be formed from a metallic material such as, for example, stainless steel.

As shown in FIG. 6, the proximal component 102 includes a base 110 and a post 112 that extends away from the base 110 to an outer end 114. A pair of locking pegs 116 extend outwardly from the post 112 adjacent to the outer end 114 to define the connector 104. As described in greater detail below, the locking pegs 116 are sized and shaped to be received in a chuck 118 of the assembly tool 22 to couple the proximal component 102 (and hence the femoral head assembly 26) to the assembly tool.

The base 110 includes a concave distal surface 120 that is shaped to match a portion of the curvature of the semi-spherical proximal surface 90 of the femoral head component 16. As shown in FIG. 5, the base 110 is positioned in a pocket 122 defined in the outer sheath 100, and the post 112 extends outwardly from the pocket 122. In the illustrative embodiment, the post 112 is aligned for assembly with the bore 98 of the femoral head component 16 to extend along a common axis 124. When the femoral stem component 14 is properly aligned with the femoral head component 16 as shown in FIG. 5, the common axis 124 is coincident with the longitudinal axis 44 of the stem trunnion 36.

As described above, the femoral head protective casing 20 also includes a distal component 106 that engages the distal surface 92 of the femoral head component 16. In the illustrative embodiment, the distal component 106 includes a number of component segments 130 that surround the opening 94 of the distal bore 98 of the femoral head component 16. Each distal component segment 130 is formed from a polymeric material such as, for example, polyethylene, in the illustrative embodiment.

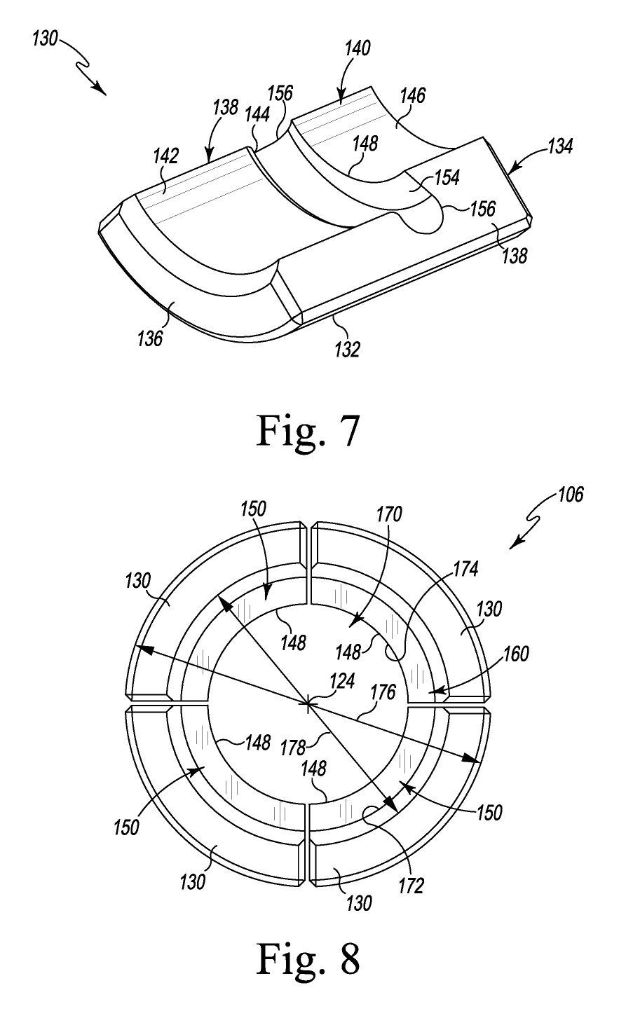

As shown in FIG. 7, each component segment 130 has a convex curved outer wall 132 that extends from a proximal end 134 to a distal end 136. The proximal end 134 of each segment 130 is a planar and is configured to engage with the distal surface 92 of the femoral head component 16. Each component segment 130 also includes a pair of side walls 138 that extend from the outer wall 132. Each side wall 138 is also planar and shaped to engage the side wall 138 of an adjacent component segment 130, as shown in FIG. 8, to form the distal component 106.

As shown in FIG. 7, each component segment 130 includes a stepped inner wall 140 that is positioned opposite the outer wall 132. The inner wall 140 extends between the pair of side walls 138 from the proximal end 134 to the distal end 136. The stepped inner wall 140 includes a concave distal surface 142 that extends inwardly from the distal end 136 to an inner edge 144. The stepped inner wall 140 also includes a concave proximal surface 146 that extends inwardly from the proximal end 134 of the component segment 130 to an inner edge 148. It should be appreciated that each of the surfaces 142, 146 has a constant radius such that, when arranged as shown in FIG. 8, the surfaces 142, 146 define a stepped cylindrical passageway. In other embodiments, however, the surfaces may be tapered.

The edges 144, 148 of the stepped inner wall 140 are spaced apart from one another such that a transverse channel 150 is defined in the inner wall 140 between the edges 144, 148. As shown in FIG. 7, the transverse channel 150 is defined by a concave curved surface 152 that extends from the edge 144 to a convex curved surface 154 that extends from the edge 148. The channel 150 extends between openings 156 defined in the side walls 138 such that when the segments 130 are assembled as shown in FIG. 8, the channels 150 cooperate to define an annular groove 160 in the distal component 106.

As shown in FIG. 8, the component segments 130 are arranged about the common axis 124 of the femoral head protective casing 20 to form the distal component 106. The stepped inner walls 140 of the component segments 130 cooperate to define a central passageway 170 extending along the axis 124 through the distal component 106. The central passageway 170 includes an outer, distal section 172 that is defined by the concave distal surfaces 142 of the segments 130 and an inner, proximal section 174 that is defined by the concave proximal surfaces 146 of the segments 130. In the illustrative embodiment, the concave proximal surfaces 146 (and hence the inner edges 148) are positioned radially inward (i.e., toward the common axis 124) from the concave distal surfaces 142 such that the outer, distal section 172 has a diameter 176 that is greater than the diameter 178 of the inner, proximal section 174. The diameter 178 is sized to correspond to the diameter of the opening 94 of the tapered bore 98 of the head component 16.

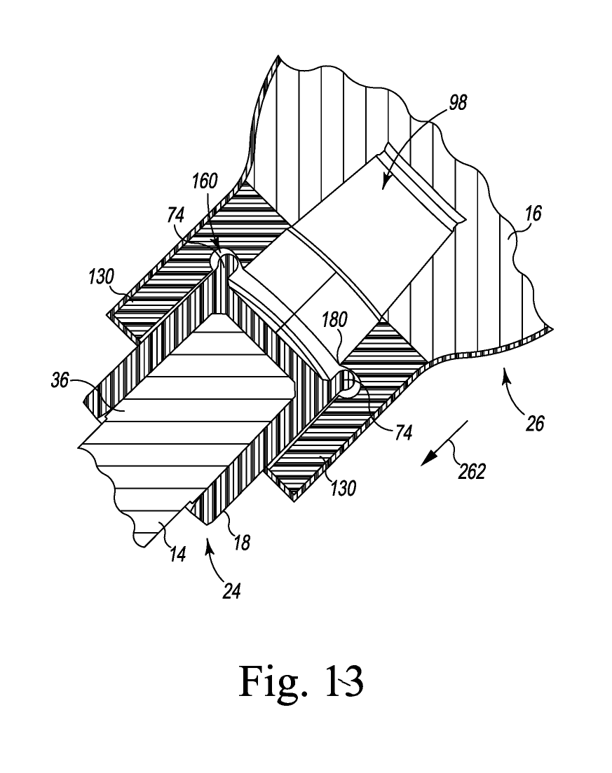

The outer, distal diameter 176 of the distal component 106 is sized to be greater than the diameter 84 defined by the proximal tabs 74 of the trunnion protective casing 18 such that the trunnion protective casing 18 may be advanced into and along the central passageway 170 to position the proximal tabs 74 in the annular groove 160 of the distal component 106 (see FIG. 13). As described in greater detail, the inner edges 148 of the component segments 130 cooperate to define an annular flange 180 that is configured to pierce the proximal end 52 of the trunnion protective casing 18 and separate the casing 18 into the segments 66, 68, 70, 72 to facilitate assembly of the stem component 14 with the head component 16.

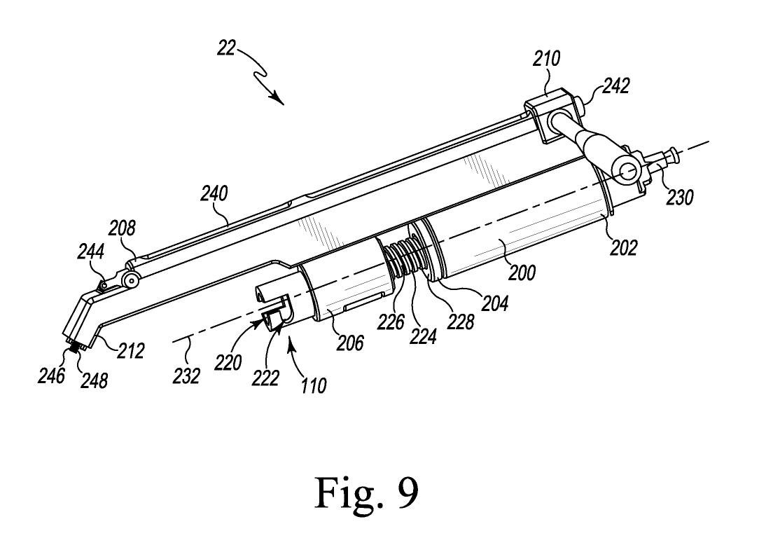

Referring now to FIG. 9, the assembly tool 22 of the orthopaedic system 10 includes a main body 200 that extends from a proximal end 202 to a distal end 204. The assembly tool 22 also includes a press frame 206 that is positioned distal of the distal end 204 of the main body 200. The main body 200 and the press frame 206 are mounted on an elongated arm 208 that extends from a proximal end 210 to a distal tip 212. As described in greater detail below, the main body 200 and the press frame 206 are configured to slide on the elongated arm 208 toward and away from the distal tip 212.

As described above, the assembly tool 22 includes a chuck 118 that is configured to be coupled to the connector 104 of the protective casing 20. As shown in FIG. 9, the chuck 118 includes an aperture 220 that is defined in the press frame 206, which is sized to receive the post 112 of the connector 104. The chuck 118 also includes a pair of locking slots 222 positioned on each side of the aperture 220, which are each sized to receive a locking peg 116 of the connector 104 to secure the protective casing 20 (and hence the femoral head assembly 26) to the assembly tool 22.

The assembly tool 22 also includes a central shaft 224 that connects the press frame 206 to the main body 200. The central shaft 224 includes a threaded section 226 that is threaded into a fixed nut 228 positioned in the main body 200. The shaft 224 extends outwardly from the proximal end 202 of the main body 200 to a tool shank 230. The tool shank 230 is configured to be coupled to a rotary power tool such as, for example, a surgical drill or T-handle, such that the tool shank 230 (and hence the central shaft 224) may be rotated about a longitudinal axis 232. In the illustrative embodiment, the central shaft 224 is rotatively coupled to the press frame 206. When the central shaft 224 is rotated about the longitudinal axis 232, the press frame 206 is configured to slide along the elongated arm 208 toward or away from the main body 200. It should be appreciated that the assembly tool 22 also includes a locking arm (not shown) that is pivotally coupled to the elongated arm 208 and is configured to engage the main body 200 to prevent the main body 200 from sliding relative to the elongated arm 208. In that way, the press frame 206 may be advance toward or away from the distal tip 212 of the elongated arm 208 independently of the main body 200.

To couple the main body 200 and the press frame 206 to the elongated arm 208, the assembly tool 22 includes a dove-tail joint. Each of the main body 200 and the press frame 206 includes a tail that is positioned in a socket defined in the elongated arm 208. It should be appreciated that in other embodiments other joints may be utilized to couple the main body 200 and the press frame 206 to the elongated arm 208.

The assembly tool 22 also includes an elongated rod 240 that is pivotally coupled to the elongated arm 208. The rod 240 includes a socket that is defined in its proximal end 242. The socket is illustratively a hex-shaped socket that is sized to receive a hex-headed wrench. The assembly tool 22 also includes a universal joint bracket 244 that is pivotally coupled to the distal end of the elongated rod 240. A threaded shaft 246 is also coupled pivotally coupled to the bracket 244. As shown in FIG. 9, the shaft 246 extends through the elongated arm 208 to a distal end 248 extending outwardly from the distal tip 212 of the arm. The shaft 246 is configured to be threaded into a threaded aperture 250 (see FIG. 2) defined in the elongated body 32 of the stem component 14. In that way, the femoral stem assembly 24 may be coupled to the assembly tool 22.

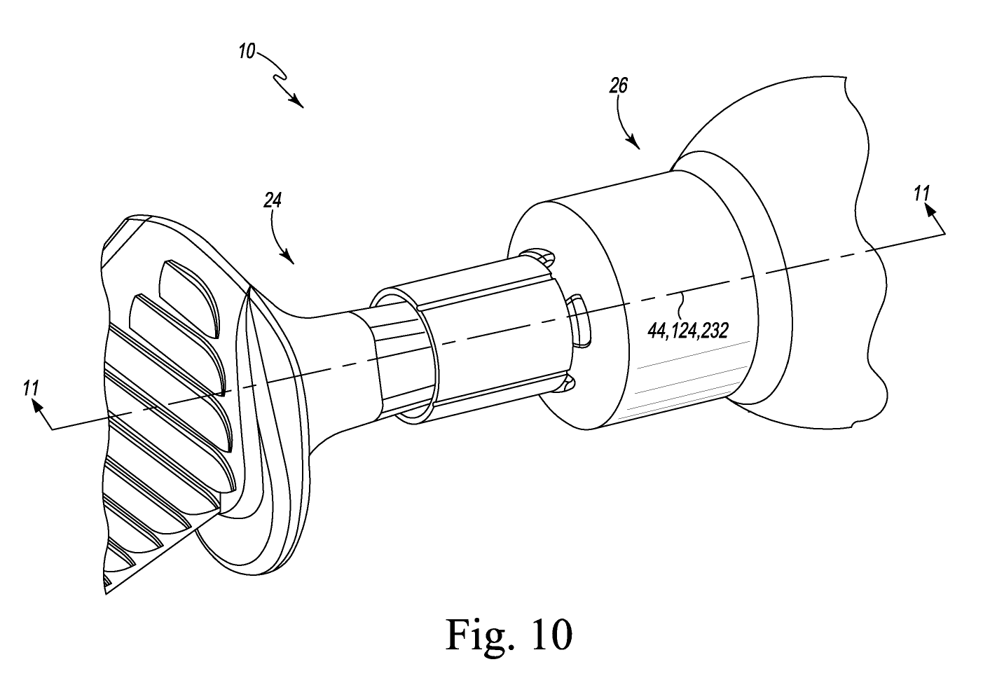

Referring now to FIGS. 10-14, a surgeon may utilize the assembly tool 22 and the femoral assemblies 24, 26 to secure a selected femoral head component 16 to a selected femoral stem component 14. This may be done either with the femoral stem component implanted in the patient or with the femoral stem component not yet implanted. It should be appreciated that during surgery, a surgeon may surgically prepare a proximal end of a patient's tibia to receive the femoral stem component 14. The surgeon may then implant the femoral stem component 14 using conventional surgical techniques. With the femoral stem component 14 extending outwardly form the proximal end of the patient's tibia, the surgeon may perform a trial reduction utilizing a femoral head trial component (not shown), which is sized to be positioned over the trunnion protective casing 18 of the femoral stem assembly 24, and an acetabular cup trial. During the trial reduction, the surgeon may evaluate the range of motion and other performance characteristics of the patient's hip joint with the trials in place to select a final femoral head component 16.

After selecting the final femoral head component 16, the surgeon may secure the femoral head assembly 26 including the selected head component 16 to the assembly tool 22. To do so, the surgeon may align the connector 104 of the femoral head assembly 26 with the chuck 118 of the assembly tool 22. The surgeon may then advance the locking pegs 116 of the connector 104 into the locking slots 222 of the chuck 118 to couple the femoral head assembly 26 to the assembly tool 22.

In the illustrative embodiment, the surgeon also secures the assembly tool 22 to the femoral stem assembly 24. To do so, the surgeon engages the threaded shaft 246 extending outwardly from the distal tip 212 of the elongated arm 208 of the assembly tool 22 with the threaded aperture 250 of the femoral stem component 14. The surgeon may then rotate the elongated rod 240 of the assembly tool 22 about its axis, thereby causing the universal joint bracket 244 to rotate. The pivotal connection between the universal joint bracket 244 and the elongated rod 240 and the threaded shaft 246 causes the threaded shaft 246 to rotate about its axis with the elongated rod 240 to thread into the aperture 250 of the femoral stem component 14, thereby securing the component 14 to the assembly tool 22.

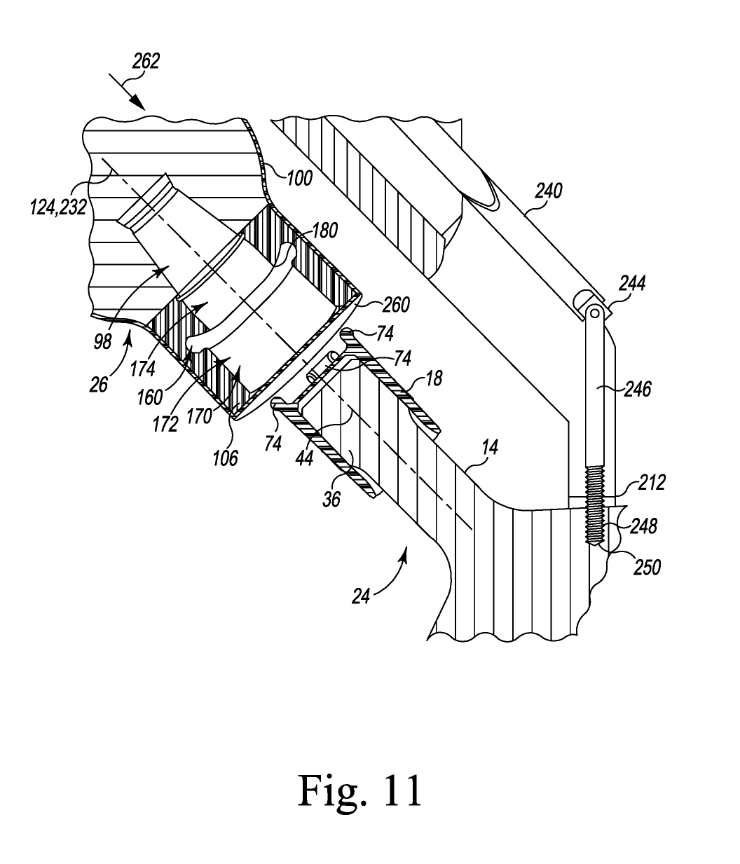

When the femoral stem assembly 24 is secured to the assembly tool 22, the trunnion axis 44 is co-incident with the axis 124 of the femoral head assembly 26 and the axis 232 of the assembly tool 22, as shown in FIG. 10 (the assembly tool is not shown in FIG. 10 for ease of reference). As shown in FIG. 11, the protective casing 18 of the femoral stem assembly 24 is aligned with the central passageway 170 of the protective casing 20. The central passageway 170 is covered by a distal section 260 of the outer sheath 100 of the casing 18. To move the protective casing 18 (and hence the trunnion 36) into the central passageway 170, the surgeon may utilize a rotary tool to rotate the tool shank 230 of the assembly tool 22 about the axis 232 and cause the press frame 206 to advance distally along the elongated arm 208 toward the femoral stem assembly 24, as indicated by arrow 262 in FIG. 11.

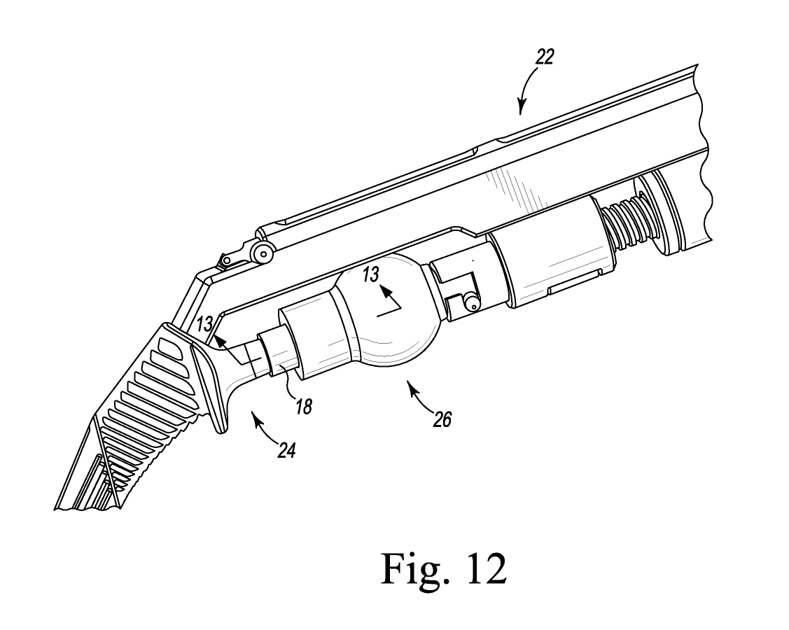

As the press frame 206 (and hence the femoral head assembly 26) move along the elongated arm 208, the proximal tabs 74 of the protective casing 18 engage the distal section 260 of the outer sheath 100 and pierce the distal section 260 to enter the central passageway 170. Continued rotation of the tool shank 230 causes the femoral head assembly 26 to continue moving distally to the position shown in FIG. 12. As shown in FIG. 12, the protective casing 18 and the trunnion 36 of the stem assembly 24 extend outwardly from the femoral head assembly 26.

As shown in FIG. 13, the proximal tabs 74 of the casing 18 are advanced into engagement with the annular flange 180 of the casing 20 and are positioned in enter the annular groove 160 surrounding the central passageway 170. When the surgeon operates the rotary tool to cause the head assembly 26 to continue moving distally, the tabs 74 are advanced radially outward by the flange 180 into the annular groove 160, and each tab 74 is captured within one of the channels 150 of each component segment 130. The leading edge 148 of the flange 180 engages the proximal end 52 of the protective casing 18, thereby cutting into the protective casing 18. Because the seams 64 define regions of less material strength than the sleeve segments 66, 68, 70, and 72, the engagement between the tabs 74 and the flange 180 causes the casing 18 to splitter or separate along the seams 64 into the sleeve segments 66, 68, 70, and 72. In that way, the seams facilitate the separation of the casing 18 into the segments to permit the casing 18 to be removed from the trunnion 36. Additionally, the engagement between the tabs 74 and the component segments 130 ensures that the splintered casing 18 remains coupled the casing 20.

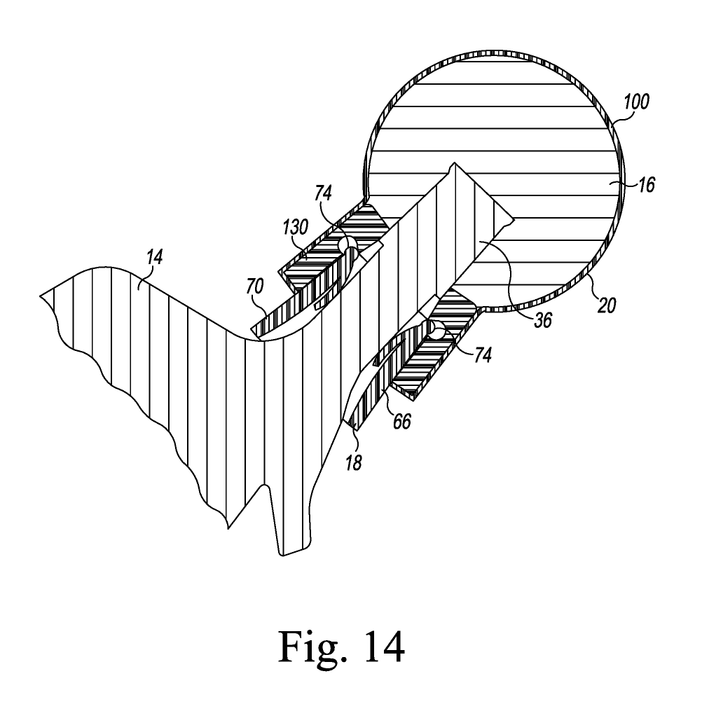

As the femoral head component 16 advances over the trunnion 36 of the femoral stem component 14, the components segments 130 (and their respective sleeve segments 66, 68, 70, or 72) spread outward away from the axes 44, 124, 232, as shown in FIG. 14. When the femoral head component 16 is taper locked to the femoral stem component 14, the surgeon may peel the splintered proactive casings 18, 20 off of the assembled components 14, 16. The engagement between the tabs 74 and the component segments 130 keeps the casings 18, 20 in one piece, which may be discarded.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic system, comprising:
a femoral head component including a semi-spherical articular surface, a distal surface, and a tapered bore formed in the distal surface,
a femoral stem component configured to be received in a proximal end of a patient's surgically-prepared femur, the stem component comprises (i) a neck including a trunnion sized to be positioned in the tapered bore of the femoral head component, and (ii) an elongated body extending distally from the neck, and
a first removable shell extending over the trunnion of the femoral stem component, the first removable shell including a number of seams extending parallel to a longitudinal axis of the trunnion, each seam being configured to facilitate separation of the first removable shell into a number of shell segments to permit the first removable shell to be removed from the trunnion,
wherein the tapered trunnion of the femoral stem component is configured to be taper locked to the femoral head component when the first removable shell is removed from the tapered trunnion.

2. The orthopaedic system of claim 1, further comprising a second removable shell coupled to the femoral head component extending over the semi-spherical articular surface and a distal opening of the tapered bore,
wherein the first removable shell includes a sleeve extending over the trunnion of the femoral stem component and a proximal tab extending outwardly from the sleeve, the proximal tab being configured to pierce the second removable shell.

3. The orthopaedic system of claim 2, wherein the second removable shell includes an inner flange configured to engage the proximal tab of the first removable shell to facilitate separation of the first removable shell into the number of shell segments.

4. The orthopaedic system of claim 3, wherein the second removable shell further includes:
a distal component coupled to the distal surface of the femoral head component, the distal component including (i) a central passageway aligned with the tapered bore of the femoral head component, the central passageway being sized to receive the first removable shell and the tapered trunnion of the femoral stem component, and (ii) the inner flange, the inner flange extending into the central passageway, and
a flexible sheath encasing the femoral head component and the distal component.

5. The orthopaedic system of claim 4, wherein the proximal tab of the first removable shell includes a plurality of proximal tabs extending from the sleeve that are configured to pierce a surface of the flexible sheath covering the central passageway, each proximal tab being connected to a section of the sleeve positioned between a pair of seams.

6. The orthopaedic system of claim 5, wherein the inner flange is an annular flange configured to engage the plurality of proximal tabs and extending around the central passageway of the distal component.

7. The orthopaedic system of claim 4, wherein the distal component of the second removable shell includes a plurality of component segments, each component segment including a pair of sidewalls extending along a longitudinal axis of the central passageway, each sidewall of each component segment being engaged with the sidewall of an adjacent component segment.

8. The orthopaedic system of claim 2, further comprising a surgical instrument configured to be coupled to the second removable shell and the elongated body of the femoral stem component, the surgical instrument being operable to advance the second removable shell distally into contact with the proximal tab of the first removable shell to pierce the second removable shell.

9. The orthopaedic system of claim 8, wherein the second removable shell includes:
a distal component coupled to the distal surface of the femoral head component, the distal component including a central passageway aligned with the tapered bore of the femoral head component, the central passageway being sized to receive the first removable shell and the tapered trunnion of the femoral stem component,
a flexible sheath encasing the femoral head component and the distal component, and
a proximal component extending outwardly from the flexible sheath, the proximal component including a concave distal surface shaped to match a curvature of the semi-spherical articular surface of the femoral head component and a connector configured to be coupled to the surgical instrument.

10. The orthopaedic system of claim 1, wherein the first removable shell includes a body having a plurality of longitudinal segments connected via the number of seams, each longitudinal segment corresponding to one of the number of shell segments.

11. The orthopaedic system of claim 10, wherein each longitudinal segment has a first thickness and each seam has a second thickness that is less than the first thickness.

12. An orthopaedic system, comprising:
a femoral head component including a semi-spherical articular surface, a distal surface, and a tapered bore formed in the distal surface, and a removable shell coupled to the femoral head component, the removable shell comprising:
a distal component coupled to the distal surface of the femoral head component, the distal component including a central passageway aligned with the tapered bore of the femoral head component,
a flexible sheath encasing the femoral head component and the distal component, and
a proximal component extending outwardly from the flexible sheath, the proximal component including a concave distal surface shaped to match a curvature of the semi-spherical articular surface of the femoral head component and a connector configured to be coupled to a surgical instrument.

13. The orthopaedic system of claim 12, wherein the distal component of the removable shell includes a plurality of component segments, each component segment including a pair of sidewalls extending along a longitudinal axis of the central passageway, each sidewall of each component segment being engaged with the sidewall of an adjacent component segment.

14. The orthopaedic system of claim 12, further comprising:
a femoral stem component configured to be received in a proximal end of a patient's surgically-prepared femur, the stem component comprises (i) a neck including a trunnion sized to be positioned in the tapered bore of the femoral head component, and (ii) an elongated body extending distally from the neck,
wherein the central passageway of the removable shell is sized to receive the trunnion of the femoral stem component.

15. The orthopaedic system of claim 14, wherein the removable shell is a femoral head protective casing, and the orthopaedic system further comprises a trunnion protective casing that extends over the trunnion of the femoral stem component, the trunnion protective casing including a proximal tab configured to pierce the femoral head protective casing to permit the trunnion and the trunnion protective casing to enter the central passageway of the trunnion protective casing.

16. The orthopaedic system of claim 15, wherein:
the trunnion protective casing includes a plurality of longitudinal segments extending over the trunnion of the femoral stem component, the longitudinal segments being connected via a number of seams, and
the proximal tab is one of a plurality of proximal tabs, each proximal tab extending proximally from a corresponding longitudinal segment.

17. The orthopaedic system of claim 16, wherein the distal component of the femoral head protective casing includes an inner flange extending into the central passageway, the inner flange being configured to engage the plurality of proximal tabs to separate the trunnion protective casing into the plurality of longitudinal segments to detach the trunnion protective casing from the trunnion.

18. A method comprising:
selecting a femoral stem component comprising (i) a neck including a tapered trunnion covered by a first removable shell and (ii) an elongated body extending distally from the neck,
selecting a femoral head component covered by a second removable shell,
aligning a distal end of the second removable shell with the tapered trunnion of the femoral stem component,
positioning the second removable shell over the first removable shell and the tapered trunnion to engage a plurality of proximal tabs of the first removable shell with an inner flange of the second removable shell,
advancing the second removable shell and the femoral head component distally to cause the proximal tabs to move distally and split the first removable shell into a number of shell segments, and
moving the femoral head component distally to position the tapered trunnion in a tapered bore of the femoral head component.

19. The method of claim 18, wherein positioning the second removable shell over the first removable shell and the tapered trunnion comprises:
piercing the distal end of the second removable shell with the plurality of proximal tabs, and
moving the first removable shell and the tapered trunnion into a central passageway defined in the second removable shell.

20. The method of claim 18, wherein advancing the second removable shell and the femoral head component distally to cause the proximal tabs to move distally and split the first removable shell into the number of shell segments further comprises separating a distal component of the second removable shell into a plurality of component segments, each component segment being coupled to a corresponding shell segment of the first removable shell.
